(19) United States
(12) Patent Application Publication
(10) Pub. No.: US 2026/0306464 A1
(43) Pub. Date: Oct. 8, 2026

(54) LINAPRAZAN GLURATE FOR TREATING GASTROESOPHAGEAL REFLUX DISEASE (GERD)

(71) Applicant: Cinclus Pharma Holding AB (publ), Stockholm (SE)

(72) Inventors: Kjell Andersson, Lindome (SE); Peter Unge, Stockholm (SE)

(21) Appl. No.: 19/482,301

(22) PCT Filed: Nov. 3, 2023
(86) PCT No.: PCT/EP2023/080637

§ 371 Date: Nov. 7, 2025

(30) Foreign Application Priority Data
May 8, 2023 (SE) 2330207-8

(51) Int. Cl. A61K 31/437; A61P 1/04

(52) U.S. Cl. CPC A61K 31/437; A61P 1/04

(57) ABSTRACT

The invention relates to linaprazan glurate, or a pharmaceutically acceptable salt thereof, for use in the treatment or prevention of a gastrointestinal inflammatory or a gastric acid related disease, such as (erosive) gastroesophageal reflux disease. The treatment involves oral administration of linaprazan glurate, or a pharmaceutically acceptable salt thereof, to a patient in an amount of from about (25) to about (100) mg once or twice daily.

### CROSS-REFERENCE TO RELATED APPLICATIONS

This application claims priority to Swedish patent application No. 2330207-8, filed May 8, 2023, the disclosure of which is incorporated herein by reference in its entirety.

### TECHNICAL FIELD

The invention relates to linaprazan glurate, or a pharmaceutically acceptable salt thereof, for use in the treatment or prevention of a gastrointestinal inflammatory or a gastric acid related disease, such as (erosive) gastroesophageal reflux disease. The treatment involves oral administration of linaprazan glurate, or a pharmaceutically acceptable salt thereof, to a patient in an amount of from about 25 to about 100 mg once or twice daily.

### BACKGROUND

Gastroesophageal reflux disease (GERD) is a digestive disease that affects the lower esophageal sphincter (the ring of muscle between the esophagus and stomach), which causes retrograde flow of gastric content into the esophagus. Symptoms of GERD include dental corrosion, dysphagia, heartburn, acid regurgitation, non-cardiac chest pain, extraesophageal symptoms such as chronic cough, hoarseness, reflux-induced laryngitis and asthma. When left untreated, GERD may result in complications such as esophagitis (inflammation of the esophagus), esophageal stricture, Barrett's esophagus (a condition involving an abnormal (metaplastic) change in the mucosal cells lining the lower portion of the esophagus), dysplasia and cancer (e.g., MALToma or adenocarcinoma).

It is estimated that 10% to 20% of the Western population is affected by GERD, but a prevalence of up to 28% has been reported in the United States (EI-Serag et al., Gut 2014, vol. 63, p. 871-880). Approximately 133 million people of the adult population in the United States and the EU-30 suffer from reflux disease. Although changes to diet and lifestyle may improve symptoms of GERD, medical treatment with proton-pump inhibitors (PPIs), H₂ receptor blockers or antacids is more effective. The global acid reflux market is dominated by PPIs, and it is estimated that more than 20% of all GERD patients take PPIs off-label twice daily to overcome the incomplete symptom relief or supplement their treatment with over-the-counter-remedies. Despite frequent off-label prescription of high dosage PPIs, many patients still suffer from poor symptom control. There is therefore a continued need for better drugs that treat GERD and provide effective relief of symptoms.

Linaprazan glurate (5-{2-[({8-[(2,6-dimethylbenzyl)amino]-2,3-dimethylimidazo[1,2-a]pyridine-6-yl}carbonyl)-amino]ethoxy}-5-oxopentanoic acid; previously known as X842) is a potassium-competitive acid blocker (P-CAB), which is another class of drugs under development for treatment of GERD. P-CABs competitively inhibit the gastric hydrogen potassium pump (H+/K+ ATPase) in the parietal cells and have a considerably faster onset to maximum effect than PPIs (typically 1-2 hours vs. 3-5 days). Neutral intragastric pH has been demonstrated two hours after the first dose with linaprazan glurate in healthy subjects. With a plasma half-life of approximately 10 hours, it is expected that administration of linaprazan glurate once or twice daily may provide 24 h acid control.

### BRIEF DESCRIPTION OF THE DRAWINGS

FIG. 1 is a plot of the healing rate (%) after 4 weeks vs. the mean percentage of time (in 24 hour) with intragastric pH>4 (based on Yuan et al., Gastroenterol. 2007, vol. 132(4), suppl. 2, A-489, No T1202).

FIG. 2 is a plot of the intragastric pH-level vs. the plasma concentration of linaprazan (nmol/L). At plasma concentrations above 240 nmol/L (the striped area), the intragastric pH is almost always above 4.

FIG. 3 provides a schematic diagram of the study design for the Linaprazan glurate Phase 1 exploratory dose-finding study.

FIG. 4 provides a schematic diagram of the study design for the Linaprazan glurate Erosive Esophagitis Dose ranging (LEED) study.

FIG. 5 is a plot of the mean LA grade improvement for LA grade C/D patients after 4 weeks of treatment with different doses of linaprazan glurate (LG) or with 30 mg QD lansoprazole (LAN).

FIG. 6 is a plot of the healing rate (%) for LA grade C/D patients after 4 weeks of treatment with different doses of linaprazan glurate (LG) or with 30 mg QD lansoprazole (LAN).

FIG. 7 is a plot of the healing rate (%) for LA grade C/D patients after 4 weeks of treatment with 75 mg BID linaprazan glurate (LG) or with 30 mg QD lansoprazole (LAN).

FIG. 8 is a plot of the healing rate (%) for all LA grade A/B/C/D patients after 4 weeks of treatment with different doses of linaprazan glurate (LG) or with 30 mg QD lansoprazole (LAN).

FIG. 9 is a chart of the mRESQ-eD assessed heartburn-free 24-hour days over a week for all patients during 8 weeks of treatment with different doses of linaprazan glurate (LG) or with 30 mg QD lansoprazole (LAN).

FIG. 10 is a chart of the mRESQ-eD assessed heartburn-free 24-hour days over a week for all LA grade A/B/C/D patients during 8 weeks of treatment with different doses of linaprazan glurate (LG) or with 30 mg QD lansoprazole (LAN).

FIG. 11 is a chart of the mRESQ-eD assessed heartburn-free 24-hour days over a week for all LA grade C/D patients during 8 weeks of treatment with different doses of linaprazan glurate (LG) or with 30 mg QD lansoprazole (LAN).

FIG. 12 shows the X-ray powder diffractogram of Form 1 of the HCl salt of linaprazan glurate, as obtained from a slurry in DMF.

FIG. 13 shows the differential scanning calorimetry (DSC) thermogram of Form 1 of the HCl salt of linaprazan glurate, as crystallized from DMF using EtOAc as the anti-solvent.

FIG. 14 shows the dynamic vapour sorption (DVS) weight change plot (A) and the DVS isotherm plot (B) for Form 1 of the HCl salt of linaprazan glurate, as crystallized from DMF using EtOAc as the anti-solvent.

### DETAILED DESCRIPTION OF THE INVENTION

In a first aspect, the invention relates to linaprazan glurate, or a pharmaceutically acceptable salt thereof, for use in the treatment or prevention of a gastrointestinal inflammatory or a gastric acid related disease, wherein linaprazan glurate, or a pharmaceutically acceptable salt thereof, is orally administered to a subject in an amount of from about 25 to about 100 mg once or twice daily.

Clinical studies have shown that treatment of eGERD with 25, 50, 75 or 100 mg of linaprazan glurate twice daily for 4 weeks resulted in high healing rates. Interestingly, it was found that such treatment provided better results than treatment with the proton pump inhibitor (PPI) lansoprazole, which currently is a standard of care treatment. As shown in the example section, 4-week treatment with linaprazan glurate resulted in a significantly higher healing rates than treatment with 30 mg/day of lansoprazole.

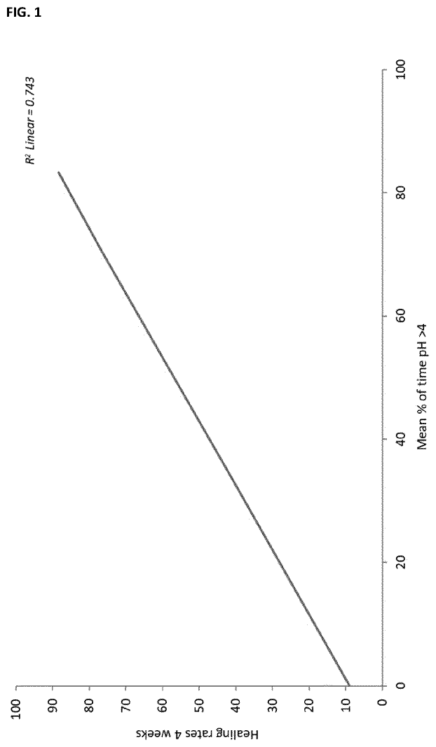

In some embodiments, linaprazan glurate, or a pharmaceutically acceptable salt thereof, is administered once daily (QD). In some embodiments, linaprazan glurate, or a pharmaceutically acceptable salt thereof, is administered twice daily (BID). It has been found that administration of linaprazan glurate twice daily results in approximately 10% longer time per day (about 2 hours) with gastric pH>4 than administration of linaprazan glurate once daily. It has previously been reported that 24-hour median pH and % of time pH>4 in 24 hour are good predictors for erosive esophagitis healing at 8 weeks, and that the better the acid suppression (pH>4), the better the efficacy in healing esophagitis (Hunt, Aliment. Pharmacol. Ther. 1995, vol. 9, suppl. 1, p. 3-7; Yuan et al., Gastroenterol. 2007, vol. 132(4), suppl. 2, A-489, No T1202). A plot of the healing rate (%) after 4 weeks vs. mean percentage of time with intragastric pH>4 is shown in FIG. 1. Administration of linaprazan glurate twice daily may therefore provide better results in the treatment of gastrointestinal inflammatory or gastric acid related diseases than administration of linaprazan glurate once daily.

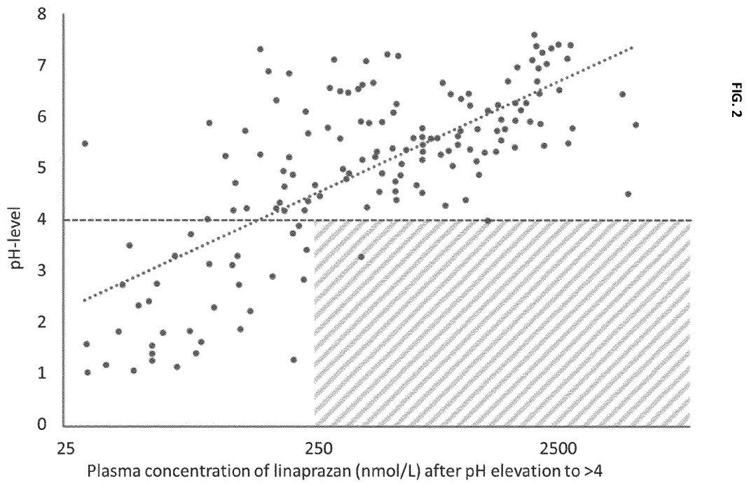

Following absorption into the blood stream, linaprazan glurate is quickly metabolized into linaprazan, which is the active metabolite. The plasma concentration of linaprazan glurate is only very low and difficult to determine, but the plasma concentration of linaprazan may be determined instead. Phase I studies have indicated that the pH control of linaprazan glurate is dose dependent, and that a minimum plasma concentration (Cmin) of linaprazan of about 240 nmol/L is necessary for maintaining the intragastric pH above 4 (see FIG. 2).

In some embodiments, therefore, the invention relates to linaprazan glurate, or a pharmaceutically acceptable salt thereof, for use in the treatment or prevention of a gastrointestinal inflammatory or a gastric acid related disease, wherein following oral administration of linaprazan glurate, or a pharmaceutically acceptable salt thereof, to a subject in an amount of from about 25 to about 100 mg once or twice daily, the subject exhibits a minimum plasma concentration (Cmin) of linaprazan of at least 240 nmol/L.

In some embodiments, linaprazan glurate, or a pharmaceutically acceptable salt thereof, is administered once or twice daily in an amount of from about 25 to about 100 mg, such as from about 25 to about 75 mg, such as from about 25 to about 50 mg, such as from about 50 to about 100 mg, such as from about 50 to about 75 mg, or such as from about 75 to about 100 mg. In some embodiments, linaprazan glurate, or a pharmaceutically acceptable salt thereof, is administered in an amount of about 25 mg once daily. In some embodiments, linaprazan glurate, or a pharmaceutically acceptable salt thereof, is administered in an amount of about 25 mg twice daily. In some embodiments, linaprazan glurate, or a pharmaceutically acceptable salt thereof, is administered in an amount of about 50 mg once daily. In some embodiments, linaprazan glurate, or a pharmaceutically acceptable salt thereof, is administered in an amount of about 50 mg twice daily. In some embodiments, linaprazan glurate, or a pharmaceutically acceptable salt thereof, is administered in an amount of about 75 mg once daily. In some embodiments, linaprazan glurate, or a pharmaceutically acceptable salt thereof, is administered in an amount of about 75 mg twice daily. In some embodiments, linaprazan glurate, or a pharmaceutically acceptable salt thereof, is administered in an amount of about 100 mg once daily. In some embodiments, linaprazan glurate, or a pharmaceutically acceptable salt thereof, is administered in an amount of about 100 mg twice daily.

In some embodiments, linaprazan glurate, or a pharmaceutically acceptable salt thereof, is administered at least 30 minutes prior to meals.

In some embodiments, linaprazan glurate, or a pharmaceutically acceptable salt thereof, is administered on demand. In some embodiments, linaprazan glurate, or a pharmaceutically acceptable salt thereof, is administered for at least 1 week. In some embodiments, linaprazan glurate, or a pharmaceutically acceptable salt thereof, is administered for at least 2 weeks. In some embodiments, linaprazan glurate, or a pharmaceutically acceptable salt thereof, is administered for at least 3 weeks. In some embodiments, linaprazan glurate, or a pharmaceutically acceptable salt thereof, is administered for at least 4 weeks. In some embodiments, linaprazan glurate, or a pharmaceutically acceptable salt thereof, is administered for at least 5 weeks, at least 6 weeks, at least 7 weeks, at least 8 weeks, at least 9 weeks or at least 10 weeks.

In some embodiments, linaprazan glurate, or a pharmaceutically acceptable salt thereof, is administered in an amount of from about 25 to about 100 mg twice daily for 4 weeks.

In some embodiments, the gastrointestinal inflammatory or gastric acid related disease is gastritis, gastroesophageal reflux disease (GERD), erosive gastroesophageal reflux disease (eGERD), H. pylori infection, Zollinger-Ellison syndrome, peptic ulcer disease (including gastric ulcers and duodenal ulcers), bleeding gastric ulcer, symptoms of gastroesophageal reflux disease (including heartburn, regurgitation and nausea), gastrinoma, acute upper gastrointestinal bleeding, or damage or bleeding caused by aspirin or NSAIDs. In some embodiments, the gastrointestinal inflammatory or gastric acid related disease is gastroesophageal reflux disease (GERD). In some embodiments, the gastrointestinal inflammatory or gastric acid related disease is erosive gastroesophageal reflux disease (eGERD).

The severity of esophagitis is typically indicated using the Los Angeles (LA) classification of reflux esophagitis, which is based on an endoscopic assessment of the patient. The LA grade scoring system divides reflux esophagitis into four categories (LA grade A to D) based on the extent of esophageal mucosal breaks. The mildest form, LA grade A esophagitis, is defined as one or more mucosal breaks not longer than 5 mm, that do not extend between the tops of two mucosal folds. LA grade B esophagitis is defined as one or more mucosal breaks longer than 5 mm that do not extend between the tops of two mucosal folds. LA grade C esophagitis is defined as one or more mucosal breaks that are continuous between the tops of two mucosal folds, but which involves less than 75% of the esophageal circumference. The most severe form, LA grade D esophagitis, is defined as one or more mucosal breaks involving at least 75% of the esophageal circumference (Lundell et al., Gut 1999, vol. 45, p. 172-180).

In some embodiments, the GERD or eGERD is LA grade A. In some embodiments, the GERD or eGERD is LA grade B. In some embodiments, the GERD or eGERD is LA grade C. In some embodiments, the GERD or eGERD is LA grade D.

In some embodiments, the subject exhibits, following oral administration of linaprazan glurate, or a pharmaceutically acceptable salt thereof, an improvement of the LA grade of reflux esophagitis. As used herein, the terms “improvement of the LA grade” and “LA grade improvement” refer to one or more steps from one LA grade to a lower LA grade (such as from grade D to grade C, B or A) or to being healed. In some embodiments, the improvement of the LA grade is 1 step. In some embodiments, the improvement of the LA grade is 2 steps. In some embodiments, the improvement of the LA grade is 3 steps. In some embodiments, the improvement of the LA grade is 4 steps.

In some embodiments, the mean LA grade improvement is at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, or at least 3.5 following oral administration of linaprazan glurate, or a pharmaceutically acceptable salt thereof, in an amount of from about 25 to about 100 mg twice daily for at least 1, 2, 3 or 4 weeks.

In some embodiments, the subject is healed following oral administration of linaprazan glurate, or a pharmaceutically acceptable salt thereof, for at least 1, 2, 3 or 4 weeks. In some embodiments, the subject is healed following oral administration of linaprazan glurate, or a pharmaceutically acceptable salt thereof, in an amount of from about 25 to about 100 mg twice daily for 4 weeks.

In some embodiments, the GERD or eGERD is LA grade A, and the subject exhibits, following oral administration of linaprazan glurate, or a pharmaceutically acceptable salt thereof, in an amount of from about 25 to about 100 mg once or twice daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 25 mg once daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 25 mg twice daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 50 mg once daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 50 mg twice daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 75 mg once daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 75 mg twice daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 100 mg once daily for at least 1, 2, 3 or 4 weeks, an LA grade improvement of 1 step. In some embodiments, the GERD or eGERD is LA grade A, and the subject exhibits, following oral administration of linaprazan glurate, or a pharmaceutically acceptable salt thereof, in an amount of about 100 mg twice daily for at least 1, 2, 3 or 4 weeks, an LA grade improvement of 1 step. An LA grade A subject exhibiting an LA grade improvement of 1 step is considered healed.

In some embodiments, the GERD or eGERD is LA grade B, and the subject exhibits, following oral administration of linaprazan glurate, or a pharmaceutically acceptable salt thereof, in an amount of from about 25 to about 100 mg once or twice daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 25 mg once daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 25 mg twice daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 50 mg once daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 50 mg twice daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 75 mg once daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 75 mg twice daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 100 mg once daily for at least 1, 2, 3 or 4 weeks, an LA grade improvement of 1 or 2 steps, more preferably 2 steps. In some embodiments, the GERD or eGERD is LA grade B, and the subject exhibits, following oral administration of linaprazan glurate, or a pharmaceutically acceptable salt thereof, in an amount of about 100 mg twice daily for at least 1, 2, 3 or 4 weeks, an LA grade improvement of 1 or 2 steps, more preferably 2 steps. An LA grade B subject exhibiting an LA grade improvement of 2 steps is considered healed.

In some embodiments, the GERD or eGERD is LA grade C, and the subject exhibits, following oral administration of linaprazan glurate, or a pharmaceutically acceptable salt thereof, in an amount of from about 25 to about 100 mg once or twice daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 25 mg once daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 25 mg twice daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 50 mg once daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 50 mg twice daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 75 mg once daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 75 mg twice daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 100 mg once daily for at least 1, 2, 3 or 4 weeks, an LA grade improvement of 1, 2 or 3 steps, more preferably 2 or 3 steps, and most preferably 3 steps. In some embodiments, the GERD or eGERD is LA grade C, and the subject exhibits, following oral administration of linaprazan glurate, or a pharmaceutically acceptable salt thereof, in an amount of about 100 mg twice daily for at least 1, 2, 3 or 4 weeks, an LA grade improvement of 1, 2 or 3 steps, more preferably 2 or 3 steps, and most preferably 3 steps. An LA grade C subject exhibiting an LA grade improvement of 3 steps is considered healed.

In some embodiments, the GERD or eGERD is LA grade D, and the subject exhibits, following oral administration of linaprazan glurate, or a pharmaceutically acceptable salt thereof, in an amount of from about 25 to about 100 mg once or twice daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 25 mg once daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 25 mg twice daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 50 mg once daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 50 mg twice daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 75 mg once daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 75 mg twice daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 100 mg once daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. In some embodiments, the GERD or eGERD is LA grade D, and the subject exhibits, following oral administration of linaprazan glurate, or a pharmaceutically acceptable salt thereof, in an amount of about 100 mg twice daily for at least 1, 2, 3 or 4 weeks, an LA grade improvement of 1, 2, 3 or 4 steps, more preferably 2, 3 or 4 steps, even more preferably 3 or 4 steps, and most preferably 4 steps. An LA grade D subject exhibiting an LA grade improvement of 4 steps is considered healed.

In some embodiments, the subject exhibits, following oral administration of linaprazan glurate, or a pharmaceutically acceptable salt thereof, a relief of reflux-related symptoms. In some embodiments, the subject exhibits an increase in 24-hour days with at most mild symptoms. In some embodiments, the subject exhibits an increase in heartburn-free 24-hour days. Relief of reflux-related symptoms may be assessed based on the modified Reflux Symptom Reflux Questionnaire-electronic Diary (mRESQ-eDiary), which is an electronic symptom diary developed for use in partial responders to proton pump inhibitors. mRESQ-eD has three domains (i.e., heartburn, other GERD signs/symptoms and regurgitations/reflux) and eight items (Andrae et al., Clin. Transl. Gastroenterol. 2020, 11(1): e00117).

In some embodiments, the subject has at least about 55% heartburn-free 24-hour days over a week, such as at least about 60%, at least about 65%, at least about 70%, at least about 75%, at least about 80%, at least about 85% or at least about 90% heartburn-free 24-hour days over a week. In some embodiments, the subject has at least about 95% heartburn-free 24-hour days over a week.

Also provided herein is a method for treating or preventing a gastrointestinal inflammatory or a gastric acid related disease in a subject in need thereof, the method comprising orally administering to the subject linaprazan glurate, or a pharmaceutically acceptable salt thereof, in an amount of from about 25 to about 100 mg once or twice daily. Also provided herein is the use of linaprazan glurate, or a pharmaceutically acceptable salt thereof, in the manufacture of a medicament for the treatment or prevention of a gastrointestinal inflammatory or a gastric acid related disease, wherein linaprazan glurate, or a pharmaceutically acceptable salt thereof, is administered orally in an amount of from about 25 to about 100 mg once or twice daily.

In some embodiments, the invention relates to a pharmaceutical formulation comprising linaprazan glurate, or a pharmaceutically acceptable salt thereof, for use in the treatment or prevention of a gastrointestinal inflammatory or a gastric acid related disease, wherein the pharmaceutical formulation comprises linaprazan glurate, or a pharmaceutically acceptable salt thereof, in an amount of from about 25 to about 100 mg and is orally administered to a subject once or twice daily.

### Formulations

Linaprazan glurate, or a pharmaceutically acceptable salt thereof, may be administered to the subject as a formulation in the form of e.g., a tablet or capsule.

A formulation of linaprazan glurate, or a pharmaceutically acceptable salt thereof, comprises a therapeutically effective amount linaprazan glurate, or a pharmaceutically acceptable salt thereof, in association with one or more pharmaceutically acceptable excipients. The excipients may e.g., include fillers, binders, surfactants, disintegrants, glidants and lubricants.

In some embodiments, the formulation comprises a surfactant. The surfactant may be a cationic surfactant, an anionic surfactant or a nonionic surfactant. Examples of cationic surfactants include, but are not limited to, cetyltrimethylammonium bromide (cetrimonium bromide) and cetylpyridinium chloride. Examples of anionic surfactants include, but are not limited to, sodium dodecyl sulfate (sodium lauryl sulfate) and ammonium dodecyl sulfate (ammonium lauryl sulfate). Examples of nonionic surfactants include, but are not limited to, glycerol monooleate, glycerol monostearate, polyoxyl castor oil (Cremophor EL), poloxamers (e.g., poloxamer 407 or 188), polysorbate 80 and sorbitan esters (Tween). In some embodiments, the surfactant is an anionic surfactant. In a preferred embodiment, the anionic surfactant is sodium dodecyl sulfate.

The surfactant may be present in the formulation in an amount of from about 1.0% (w/w) relative to the amount of linaprazan glurate, or a pharmaceutically acceptable salt thereof, such as from about 2.0 to about 12.0% (w/w), such as from about 4.0 to about 12.0% (w/w), such as from about 6.0 to about 12.0% (w/w), such as from about 8.0 to about 12.0% (w/w), or such as from about 10.0 to about 12.0% (w/w) relative to the amount of linaprazan glurate, or a pharmaceutically acceptable salt thereof. The amount of surfactant in the formulation is preferably as low as possible. Therefore, in another embodiment, the formulation comprises from about 1.0 to about 11.0% (w/w), such as from about 1.0 to about 10.0% (w/w), such as from about 1.0 to about 9.0% (w/w), such as from about 1.0 to about 8.0% (w/w), or such as from about 1.0 to about 7.0% (w/w) of surfactant relative to the amount of linaprazan glurate, or a pharmaceutically acceptable salt thereof. In another embodiment, the formulation comprises from about 2.0 to about 11.0% (w/w), such as from about 2.0 to about 10.0% (w/w), such as from about 2.0 to about 9.0% (w/w), such as from about 2.0 to about 8.0% (w/w), or such as from about 2.0 to about 7.0% (w/w) of surfactant relative to the amount of linaprazan glurate, or a pharmaceutically acceptable salt thereof. In yet another embodiment, the formulation comprises from about 4.0 to about 11.0% (w/w), such as from about 4.0 to about 10.0% (w/w), such as from about 4.0 to about 9.0% (w/w), such as from about 4.0 to about 8.0% (w/w), or such as from about 4.0 to about 7.0% (w/w) of surfactant relative to the amount of linaprazan glurate, or a pharmaceutically acceptable salt thereof.

In some embodiments, the formulation comprises surfactant in an amount of about 11.0% (w/w) or less, relative to the amount of linaprazan glurate, or a pharmaceutically acceptable salt thereof. In some embodiments, the formulation comprises surfactant in an amount of about 10.0% (w/w) or less, relative to the amount of linaprazan glurate, or a pharmaceutically acceptable salt thereof. In some embodiments, the formulation comprises surfactant in an amount of about 9.0% (w/w) or less, relative to the amount of linaprazan glurate, or a pharmaceutically acceptable salt thereof. In some embodiments, the formulation comprises surfactant in an amount of about 8.0% (w/w) or less, surfactant relative to the amount of linaprazan glurate, or a pharmaceutically acceptable salt thereof.

In some embodiments, the formulation comprises a filler. Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch. In some embodiments, the filler is lactose, such as lactose monohydrate.

In some embodiments, the formulation comprises a binder. Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum and tragacanth gum), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)).

In some embodiments, the formulation comprises a disintegrant. Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)). In some embodiments, the disintegrant is croscarmellose sodium.

In some embodiments, the formulation comprises a glidant or lubricant. Examples of suitable glidants and lubricants include, but are not limited to, talc, magnesium stearate, calcium stearate, sodium stearyl fumarate, stearic acid, glyceryl behenate, colloidal anhydrous silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil. In certain embodiments, the glidant is colloidal anhydrous silica.

Pharmaceutical formulations of linaprazan glurate have been disclosed in e.g., WO 2021/089580 and WO 2023/185624. In some embodiments, a tablet formulation of linaprazan glurate has the composition as shown in table 1:

TABLE 1
Tablet formulation of linaprazan glurate

| Ingredient | Amount (%) |
|---|---|
| Active pharmaceutical ingredient | |
| Linaprazan glurate | 9.09 |
| Excipients | |
| Lactose monohydrate | 84.7 |
| Croscarmellose sodium | 4.00 |
| Sodium dodecyl sulfate | 1.00 |
| Sodium stearyl fumarate | 1.00 |
| Colloidal anhydrous silica | 0.25 |
| Total | 100 |

In general, formulations may be prepared in a conventional manner using conventional excipients. In some embodiments, the ingredients of the formulation are mixed to a homogenous mixture and then formulated as tablets or capsules. The homogenous mixture of the ingredients may be compressed into tablets using conventional techniques, such as a rotary tablet press. The mixture of ingredients may also be granulated. For instance, the mixture of ingredients may be wetted by the addition of a liquid, such as water and/or an appropriate organic solvent (e.g., ethanol or isopropanol), and thereafter granulated and dried. Alternatively, granules may be prepared by dry granulation, such as by roller compaction. The granules obtained may be compressed into tablets using conventional techniques. Capsules may comprise a powder mixture or small multiparticulates (such as granules, extruded pellets or minitablets) of the ingredients. If desirable, any of the tablets, capsules, granules, extruded pellets and minitablets mentioned above may be coated with one or more coating layers. Such coating layers may be applied by methods known in the art, such as by film coating involving perforated pans and fluidized beds. In some embodiments, the formulation is in the form of a tablet.

### Salts and Crystalline Forms

In some embodiments, linaprazan glurate, or a pharmaceutically acceptable salt thereof, is present in crystalline form. In some embodiments, linaprazan glurate is present as a crystalline anhydrate, such as disclosed in US 2022/0002297. In a particular embodiment, the crystalline anhydrate of linaprazan glurate is Form A.

In a specific embodiment, Form A has an X-ray powder diffraction (XRPD) pattern, obtained with CuKα1-radiation, with at least peaks at ° 2θ values of 9.9±0.2 and 11.5±0.2. In a more specific embodiment, Form A has an X-ray powder diffraction pattern, obtained with CuKα1-radiation, with at least peaks at 9.9±0.2 and 11.5±0.2 and one or more of 8.4±0.2, 15.5±0.2 and 16.8±0.2.

In a more specific embodiment, Form A has an XRPD pattern, obtained with CuKα1-radiation, with at least peaks at ° 2θ values of 8.4±0.2, 9.9±0.2, 11.5±0.2, 15.5±0.2, 16.8±0.2, 23.5±0.2, 24.9±0.2 and 25.5±0.2.

In a more specific embodiment, Form A has an XRPD pattern, obtained with CuKα1-radiation, with at least peaks at ° 2θ values of 8.4±0.2, 9.9±0.2, 11.5±0.2, 15.5±0.2, 16.8±0.2, 23.5±0.2, 24.9±0.2 and 25.5±0.2, and one or more of 18.2±0.2, 18.4±0.2, 21.0±0.2, 21.2±0.2 and 23.3±0.2.

In some embodiments, linaprazan glurate is present as a pharmaceutically acceptable salt of linaprazan glurate. Suitable examples of pharmaceutically acceptable salts of linaprazan glurate include, but are not limited to, a hydrochloride salt, a hydrobromide salt, a methanesulfonic acid salt (“mesylate”) or a maleic acid salt (“maleate”). In some embodiments, linaprazan glurate is present as a hydrochloride salt of linaprazan glurate.

In some embodiments, linaprazan glurate is present as a crystalline hydrochloride salt of linaprazan glurate, such as disclosed in WO 2023/079094.

In some embodiments, linaprazan glurate is present as a crystalline HCl salt of linaprazan glurate, which is stable at a relative humidity (RH) of 94% at room temperature. Such crystalline HCl salts can be stable under these conditions for at least 1 day, 1 week, 1 month, 3 months, 6 months, 1 year, 2 years, 3 years or even longer.

In some embodiments, linaprazan glurate is present as a crystalline anhydrate of the HCl salt of linaprazan glurate. In a particular embodiment, the crystalline anhydrate of the HCl salt of linaprazan glurate is Form 1. This stable form may be prepared directly from the free base of linaprazan glurate, or by certain crystallisation techniques using the hydrochloride salt thereof, e.g. from a slurry in DMF, pyridine, benzyl alcohol or ethanol; by anti-solvent crystallisation from DMF or pyridine and certain anti-solvents; or by cooling from DMF or pyridine.

In a specific embodiment, Form 1 of the HCl salt of linaprazan glurate has an X-ray powder diffraction (XRPD) pattern, obtained with CuKα1-radiation, with at least two peaks at ° 2θ values selected from the list consisting of 9.1±0.2, 13.8±0.2, 14.0±0.2, 20.0±0.2, 22.9±0.2, 23.4±0.2, 24.4±0.2, 24.6±0.2 and 26.7±0.2.

In a more specific embodiment, Form 1 of the HCl salt of linaprazan glurate has an XRPD pattern, obtained with CuKα1-radiation, with at least peaks at ° 2θ values of 20.0±0.2 and 26.7±0.2.

In a more specific embodiment, Form 1 of the HCl salt of linaprazan glurate has an XRPD pattern, obtained with CuKα1-radiation, with at least four peaks at ° 2θ values selected from the list consisting of 9.1±0.2, 13.8±0.2, 14.0±0.2, 20.0±0.2, 22.9±0.2, 23.4±0.2, 24.4±0.2, 24.6±0.2 and 26.7±0.2.

In a more specific embodiment, Form 1 of the HCl salt of linaprazan glurate has an XRPD pattern, obtained with CuKα1-radiation, with at least peaks at ° 2θ values of 20.0±0.2, 24.4±0.2, 24.6±0.2 and 26.7±0.2.

In a more specific embodiment, Form 1 of the HCl salt of linaprazan glurate has an XRPD pattern, obtained with CuKα1-radiation, with at least peaks at ° 2θ values of 20.0±0.2, 24.4±0.2, 24.6±0.2 and 26.7±0.2, and or more of 9.1±0.2, 13.8±0.2, 14.0±0.2, 22.9±0.2 and 23.4±0.2.

In a more specific embodiment, Form 1 of the HCl salt of linaprazan glurate has an XRPD pattern, obtained with CuKα1-radiation, with at least peaks at ° 2θ values of 9.1±0.2, 13.8±0.2, 20.0±0.2, 23.4±0.2, 24.4±0.2, 24.6±0.2 and 26.7±0.2.

In a more specific embodiment, Form 1 of the HCl salt of linaprazan glurate has an XRPD pattern, obtained with CuKα1-radiation, with at least peaks at ° 2θ values of 9.1±0.2, 13.8±0.2, 14.0±0.2, 20.0±0.2, 22.9±0.2, 23.4±0.2, 24.4±0.2, 24.6±0.2 and 26.7±0.2.

In a more specific embodiment, Form 1 of the HCl salt of linaprazan glurate has an XRPD pattern, obtained with CuKα1-radiation, with at least peaks at ° 2θ values of 9.1±0.2, 13.8±0.2, 14.0±0.2, 20.0±0.2, 22.9±0.2, 23.4±0.2, 24.4±0.2, 24.6±0.2 and 26.7±0.2, and one or more of 16.2±0.2, 18.6±0.2, 22.2±0.2, 25.6±0.2 and 27.9±0.2.

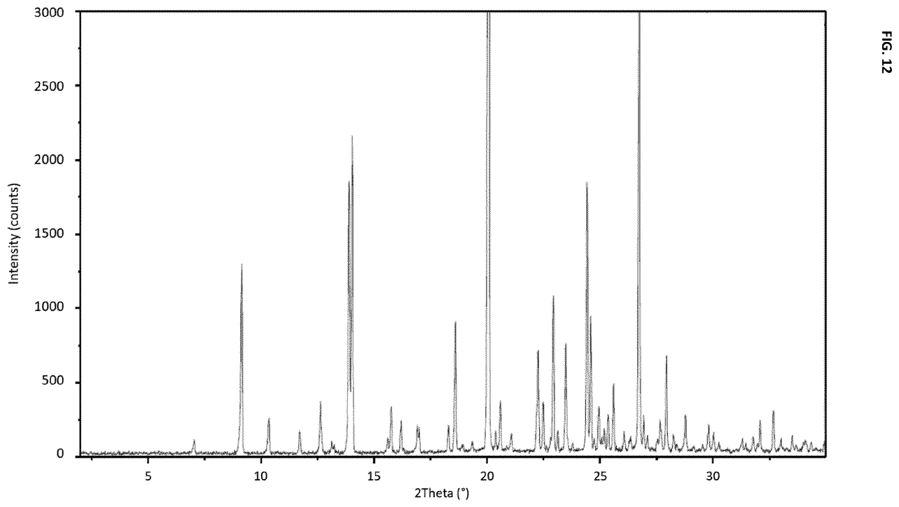

In another embodiment, Form 1 of the HCl salt of linaprazan glurate has an XRPD pattern, obtained with CuKα1-radiation, substantially as shown in FIG. 12.

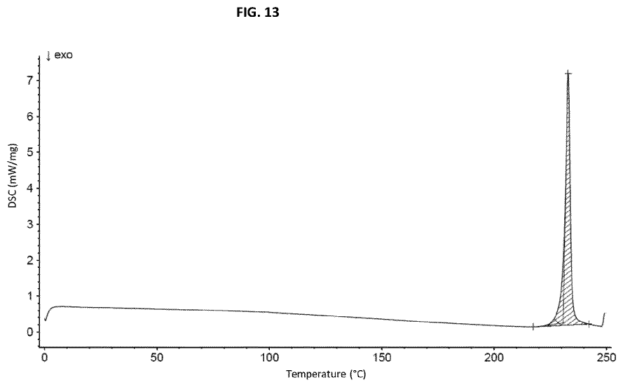

In another embodiment, Form 1 of the HCl salt of linaprazan glurate has a DSC curve comprising an endotherm between about 230° C. and about 240° C. In a particular embodiment, Form 1 of the HCl salt of linaprazan glurate has a DSC curve comprising an endotherm at approximately 233° C. The DSC thermogram of Form 1 of the HCl salt of linaprazan glurate is shown in FIG. 13.

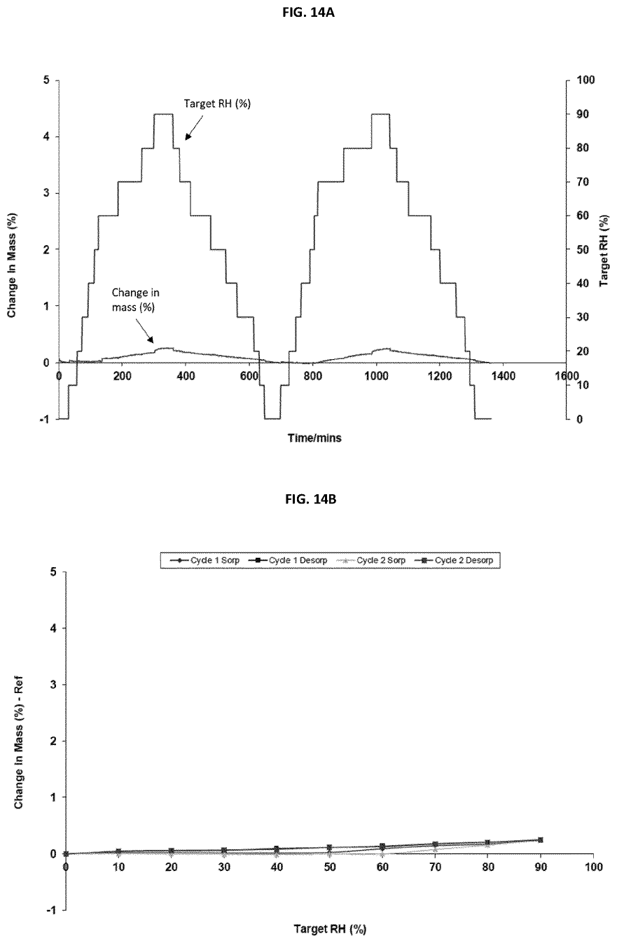

Dynamic vapour sorption analysis has shown that Form 1 of the HCl salt of linaprazan glurate has a very low hygroscopicity, with a water uptake of only about 0.2% at 90% RH. The weight change plot and the sorption isotherm plot for Form 1 of the HCl salt of linaprazan glurate showed only a small uptake of water at elevated humidities; see FIGS. 14A and 14B, respectively. This low hygroscopicity is considered advantageous, as the water content of the crystals remains substantially constant even with humidity changes within the normal relative humidity range of about 30% to about 80% RH. In some embodiments, Form 1 of the HCl salt of linaprazan glurate is stable at a relative humidity up to 90% at a temperature of 25° C.

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the terms “effective amount” or “therapeutically effective amount” refer to a sufficient amount of linaprazan glurate, or a pharmaceutically acceptable salt thereof, that, following administration to a subject, will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic use is the amount of linaprazan glurate, or a pharmaceutically acceptable salt thereof, required to provide a clinically significant decrease in disease symptoms. An appropriate “effective” amount in any individual case is determined using any suitable technique, such as a dose escalation study.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “subject,” “individual,” or “patient,” used interchangeably, refer to any animal, including mammals such as mice, rats, other rodents, rabbits, dogs, cats, swine, cattle, sheep, horses, primates, and humans. In some embodiments, the subject is a human.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the terms “twice daily” or “BID” (bis in die) refer to administration of a drug at two different times during the day, such as separated by at least about 1 hour, about 2 hours, about 3 hours, about 4 hours, about 5 hours, about 6 hours, about 7 hours, about 8 hours, about 9 hours, about 10 hours, about 11 hours, or about 12 hours. In some embodiments, twice daily refers to once in the morning and once in the evening. Administration of two or more unit doses (e.g., pills, tablets or capsules) of a drug at a single time during the day is considered once daily administration, whereas administration of two or more unit doses (e.g., pills, tablets or capsules) of a drug at two different times during the day is considered twice daily administration.

As used herein, the term “about” refers to a value or parameter herein that includes (and describes) embodiments that are directed to that value or parameter per se. For example, description referring to “about 20” includes description of “20”. Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

As used herein, the terms “crystalline form” or “polymorph” refer to crystals of the same molecule that have different physical properties as a result of the order of the molecules in the crystal lattice. Polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bioavailability), solubility, melting point, chemical stability, physical stability, powder flowability, water sorption, compaction, and particle morphology. Differences in stability can result from changes in chemical reactivity (e.g. differential oxidation, such that a dosage form discolours more rapidly when comprised of one polymorph than when comprised of another polymorph) or mechanical changes (e.g., crystal changes on storage as a kinetically favoured polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph” does not include amorphous forms of the compound.

As used herein, the term “anhydrate” or “anhydrous form” refers to a crystalline form of linaprazan glurate, or a pharmaceutically acceptable salt thereof, that has 0.5% or less by weight water, for example 0.4% or less, or 0.3% or less, or 0.2% or less, or 0.1% or less by weight water.

As used herein, the term “stable” means that the crystalline forms (i.e., polymorphs) do not exhibit a change in one or more of polymorph form (e.g., an increase or decrease of a certain form), appearance, pH, percent impurities, activity (as measured by in vitro assays), or osmolarity over time. In some embodiments, the polymorphs provided herein are stable for at least 1, 2, 3 or 4 weeks. For example, the polymorphs do not exhibit a change in one or more of polymorph form (e.g., an increase or decrease of a certain form), appearance, pH, percent impurities, activity (as measured by in vitro assays), or osmolarity over at least 1, 2, 3 or 4 weeks. In some embodiments, the polymorphs provided herein are stable for at least 1, 2, 3, 4, 5, 6, 7, 8, 9, 10, 11 or 12 months. For example, the polymorphs do not exhibit a change in one or more of polymorph form (e.g., an increase or decrease of a certain form), appearance, pH, percent impurities, activity (as measured by in vitro assays), or osmolarity over at least 1, 2, 3, 4, 5, 6, 7, 8, 9, 10, 11 or 12 months. In the above, the phrase “do not exhibit a change” refers to a change of less than 5% (e.g., less than 4%, less than 3%, less than 2%, less than 1%) as measured for any of the parameters over the relevant time period.

The invention will now be described by the following examples which do not limit the invention in any respect. All cited documents and references are incorporated by reference.

### EXAMPLES

### Example 1

### Phase 1 Exploratory Dose-Finding Study

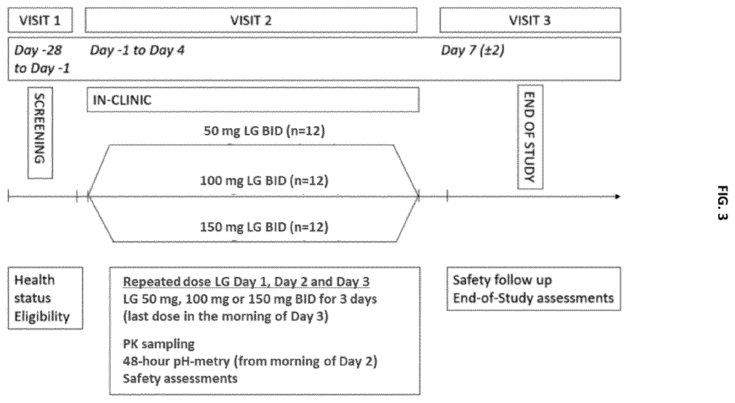

The study was a single-centre, open-label, parallel-group, randomised exploratory dose-finding study designed to evaluate the PK, PD, safety and tolerability of repeated oral doses of linaprazan glurate (LG) at 3 dose levels, in healthy male and female subjects. About 36 subjects were randomised for an estimated total of 12 evaluable subjects per treatment arm. The study design is shown in FIG. 3.

Subjects came to the clinic for 3 visits. The screening (Visit 1) took place from Day −28 to Day −1 and included an eligibility check and review of health status. At Visit 2, subjects were admitted to the clinic on Day −1 and remained at the clinic until Day 4 for repeated dose of LG administration and safety, PK and PD assessments. The subjects fasted for at least 8 hours before the anticipated dosing time on Day 1. Water, but no other drinks, was allowed as desired except for 1 hour before and 30 minutes after dosing. In the morning of Day 1, subjects were randomised to one of 3 treatment arms:
LG 50 mg BID for 3 days (5 doses, last dose in the morning of Day 3)
LG 100 mg (2×50 mg) BID for 3 days (5 doses, last dose in the morning of Day 3)
LG 150 mg (3×50 mg) BID for 3 days (5 doses, last dose in the morning of Day 3)

Subjects were carefully monitored by clinical staff during and after dosing. Subjects had a 48-hour pH-metry assessment during Day 2-4 of treatment. The pH-metry assessment was started prior to Dose 3 (in the morning of Day 2) and continued for 48 hours, until 24 hours post last dose. Vital signs and ECG were checked at regular intervals and PK sampling was done at selected timepoints. A final end-of-study visit (Visit 3) took place on Day 7 (±2) or after early withdrawal. Each subject was expected to participate in the study for approximately 7 days from the day of randomisation (Day 1, Visit 2) to the End-of Study Visit. The Screening Visit was performed within 28 days prior to randomisation (Day −28 to Day −1). The results of the pH-metry assessment are shown in tables 2 and 3 below.

TABLE 2
Administration of 2 doses (Day 2)

| | Hours with | | Percentage of | Percentage of time |
|---|---|---|---|---|
| Treatment | pH >4 | Hours with pH >4 | time with pH >4 | with pH >4 |
| Group | (Mean (SD)) | (Median (min, max)) | (Mean) | (Median (min, max)) |
| 50 mg | 15.47 (4.83) | 15.65 (8.5, 23.8) | 64.48 | 65.20 (35.4, 99.1) |
| 100 mg | 18.93 (4.83) | 21.30 (7.0, 22.8) | 78.85 | 88.70 (29.1, 95.0) |
| 150 mg | 22.00 (2.60) | 23.05 (16.6, 24.0) | 91.64 | 96.05 (69.3, 99.8) |

TABLE 3
Administration of 1 last dose in the morning (Day 3, “QD”)

| | Hours with | | Percentage of | Percentage of time |
|---|---|---|---|---|
| Treatment | pH >4 | Hours with pH >4 | time with pH >4 | with pH >4 |
| Group | (Mean (SD)) | (Median (min, max)) | (Mean) | (Median (min, max)) |
| 50 mg | 17.04 (5.33) | 15.80 (6.8, 23.8) | 71.15 | 65.90 (28.6, 99.8) |
| 100 mg | 18.38 (3.84) | 19.30 (11.6, 23.5) | 76.81 | 80.50 (48.9, 97.8) |
| 150 mg | 20.38 (3.51) | 21.05 (15.5, 24.0) | 85.14 | 88.00 (64.9, 99.8) |

For the treatment groups receiving 100 or 150 mg linaprazan glurate, administration of two daily doses (Day 2, BID) provided about two additional hours with pH>4 (median), or about 10% longer time with pH>4, than administration of a dose once daily (Day 3, “QD”). The small number of patients and the wide spread of the measured values did not allow any clear conclusions to be drawn for the 50 mg treatment group.

### Example 2

### Linaprazan Glurate Erosive Esophagitis Dose Ranging (LEED) Study

### Study Summary

The study included patients with erosive esophagitis (eGERD). Patients were divided into two cohorts, one with patients having moderate to severe eGERD (LA grade C/D) and one with patients having milder eGERD (LA grade A/B) with a history of only partial response to PPI-treatment. The primary endpoint of the study was to support dose selection of linaprazan glurate for the phase III program in eGERD, through central assessment of healing of erosive esophagitis after four weeks of treatment. The sample size, i.e., the number of patients needed, for measuring dose related efficacy was based on the patient cohort with moderate to severe eGERD. After initial endoscopy and other screening procedures, 248 patients were randomized equally into one of five treatment groups, receiving either four weeks treatment with linaprazan glurate in four dose levels, or four weeks lansoprazole in the approved standard eGERD healing dose. Thereafter, an endoscopic evaluation of healing was performed, where healing was defined as no presence of esophageal erosions, i.e., no erosive damage to the esophageal mucosa. After the endoscopic evaluation, all patient groups received lansoprazole for four weeks in a maintenance phase. A retrospective central review of the endoscopy findings was performed, and 162 patients were available for evaluation of the primary endpoint. All enrolled 248 patients were included in the safety analysis.

### Methods

This multicenter study (ClinicalTrials.gov identifier: NCT05055128) was conducted at 42 sites in Europe and the United States. The study was performed in accordance with the protocol and ethical principles that have their origin in the Declaration of Helsinki and are consistent with the International Conference on Harmonization (ICH) guidelines for Good Clinical Practice (GCP) E6 (R2), EU Clinical Trials Directive, and applicable local regulatory requirements. Patients provided written informed consent before entering the study.

### Study Design and Treatment

The study was a randomized, double-blind, active comparator-controlled study with a parallel group design including four arms with linaprazan glurate and one arm with lansoprazole. Pharmacokinetic (PK) blood sampling pre-dose, close before the first and the second dose administration was performed at Day 7, Day 14 and Day 28 visits days in all patients. 248 patients with erosive esophagitis due to GERD LA grade C or D and patients with erosive esophagitis due to GERD LA grade A or B with at least partial symptom response but still endoscopically unhealed after 8 weeks history of standard treatment healing course with PPI, were randomized.

Randomization to one of the treatments with linaprazan glurate 25 mg, 50 mg, 75 mg, 100 mg twice daily (BID), or lansoprazole 30 mg once daily (QD) was based on a 1:1:1:1:1 scheme. One site could not enroll more than about 20% of the overall number of patients (48 patients per site).

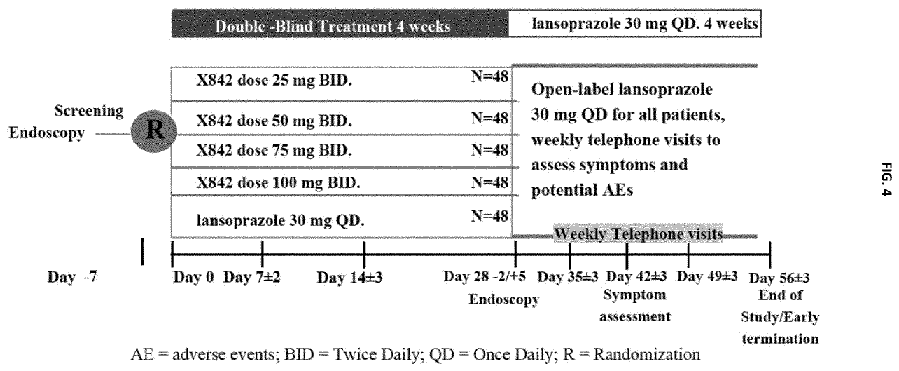

The duration of each patient's participation in the study, including screening, blind treatment period and open-label treatment period, was approximately 60 days. The patients were randomized for 4 weeks (28 days, −2/+5 days) of double-blind treatment and were provided with IMP for 35 days (including one additional blister pack dispensed at Visit 2/Day 0) to allow for treatment up to the visit window. All but twenty patients had an endoscopic evaluation after 4 weeks of treatment. Following the endoscopic evaluation, all patients received subsequent 4 weeks of open-label treatment with lansoprazole 30 mg QD. Repeated symptom evaluation to detect symptom pattern was assessed during this period. The study design is shown in FIG. 4.

Four doses of linaprazan glurate (25 mg, 50 mg, 75 mg, 100 mg, and dummies) were administered as tablets twice daily. The tablet formulation as shown in table 1 was used. The active comparator lansoprazole 30 mg and its dummy were administered as capsules once daily in the morning. The study drugs were taken with 100 mL of noncarbonated water at least 30 minutes prior to meals. The duration of double-blind treatment was 28 days −2/+5 days. To blind treatment, each patient received 2 tablets (containing doses of linaprazan glurate (LG) or its dummy) and one capsule (containing lansoprazole or its dummy) in the morning and 2 tablets (containing doses of linaprazan glurate (LG) or its dummy) in the evening, according to the scheme below:

| Randomization | | Morning | Morning | Morning | Evening | Evening |
|---|---|---|---|---|---|---|
| ratio | Treatment group | capsule | tablet 1 | tablet 2 | tablet 1 | tablet 2 |
| 1 | LG (BID) 25 mg | placebo | 25 mg | placebo | 25 mg | placebo |
| 1 | LG (BID) 50 mg | placebo | 50 mg | placebo | 50 mg | placebo |
| 1 | LG (BID) 75 mg | placebo | 50 mg | 25 mg | 50 mg | 25 mg |
| 1 | LG (BID) 100 mg | placebo | 50 mg | 50 mg | 50 mg | 50 mg |
| 1 | Lansoprazole (QD) 30 mg | 30 mg | placebo | placebo | placebo | placebo |

The study comprised 6 visits at the site and 3 telephone visits. Based on the endoscopy findings and/or signed informed consent form (ICF), the screening visit (Visit 1) took place not more than 7 working days prior to randomization and the first dose administration (Visit 2). All patients then visited the study clinic/investigational site after 7 (Visit 3), 14 (Visit 4) and 28 days (Visit 5) and at the End of Study/Early Termination visit (Visit 9). There were 3 weekly telephone visits after Visit 5.

### Patients

### Inclusion Criteria

Male or female patient aged 18-75 years inclusive at the time of obtaining the informed consent.
Body mass index (BMI)≥18 and 40 kg/m² at screening
Gastroesophageal reflux disease with endoscopically confirmed erosive esophagitis:
   LA grade C or D≤7 working days before randomization (with or without historical PPI treatment)
   or
   LA grade A or B≤7 working days before randomization
   and
      history of treatment with standard healing course of PPI (all PPIs except lansoprazole) for minimum of 8 weeks prior to screening and 7 working days of non-treatment during this period
      and
      at least partial symptom response during the minimum of 8 weeks of PPI treatment.
Willing and able to comply with all aspects of the protocol (including PK sampling, capsule swallowing, diary completion, etc.)

Exclusion criteria included the following:
History or presence of any clinically significant cardiovascular, respiratory, hepatic, renal, gastrointestinal, endocrine, hematological, or neurological disease or disorder.
Patients with so-called “alarm features” in symptomatology, like odynophagia, severe dysphagia, bleeding, weight loss, anemia, and blood in stool pointing to a possible malignant disease of the gastrointestinal (GI) tract.
Present clinically significant psychiatric diagnosis.
History of malignancy of any organ system.
Presence of esophageal ulcer, stricture, Barrett's esophagus or suspected esophagitis secondary to infection, inflammatory disease, ingestion of erosive chemicals or history of any surgical or medical condition which might significantly alter the GERD status or the absorption, distribution, metabolism or excretion of drugs.
Known severe atrophic gastritis.
Any planned major surgery within the duration of the study.
Any clinically significant laboratory parameter outside reference value that may suggest a new or insufficiently understood disease, may present an unreasonable risk to the patient, or may interfere with study assessments. Any of the following screening laboratory test are exclusionary:
   Serum ALT or AST>1.5× the upper limit of normal (ULN) for the central laboratory conducting the test;
   Serum Total Bilirubin >1.5×ULN for the central laboratory conducting the test;
   Serum Creatinine >1.5×ULN for the central laboratory conducting the test;
   Estimated glomerular filtration rate 59 mL/min (calculated using the Modification of Diet in Renal Disease equation).
History of a positive result for human immunodeficiency virus (HIV), Hepatitis B surface antigen (HBsAg), antibody to Hepatitis B core antigen (anti-HBcAg), or antibody to Hepatitis C virus (anti-HCV) or presence of these findings on screening.
After 10 min supine rest at the time of screening, any vital signs values outside the following ranges:
   Systolic blood pressure (BP)>160 mmHg or <90 mmHg
   Diastolic BP>100 mmHg or <60 mmHg
   Heart rate <40 or >95 beats per min
History of long QTc syndrome (e.g. QTc≥450 ms for males and 470 ms for females).
Cardiac arrhythmias or any clinically significant abnormalities in the resting 12-lead ECG at the time of screening, as judged by the Investigator.
History of severe allergy/hypersensitivity or ongoing allergy/hypersensitivity.
Current or history of alcohol, drug abuse and/or use of anabolic steroids within 2 years prior to screening.
Women who are pregnant or breastfeeding.

### Assessments

The primary objective of the study was to support dose selection for linaprazan glurate, through assessment of healing of erosive esophagitis due to GERD after 4 weeks of treatment. Healing of erosive esophagitis due to GERD was based on endoscopic assessment after 4 weeks of double-blind treatment.

A secondary objective of the study was the safety and tolerability of the four dose levels of linaprazan glurate and lansoprazole, with lansoprazole serving as the active comparator. Another secondary objective of the study was the reflux related symptom pattern during the initial 4 weeks treatment with four dose levels of linaprazan glurate and with lansoprazole, and the symptom pattern during the subsequent additional 4 weeks (weeks 5-8) open-label treatment with lansoprazole 30 mg QD. Heartburn-free 24-hour days and 24-hour days with at most mild heartburn symptoms were assessed based on modified eDiary (mRESQ-eD), including splits into day- and nighttime evaluations.

### Results

### Healing

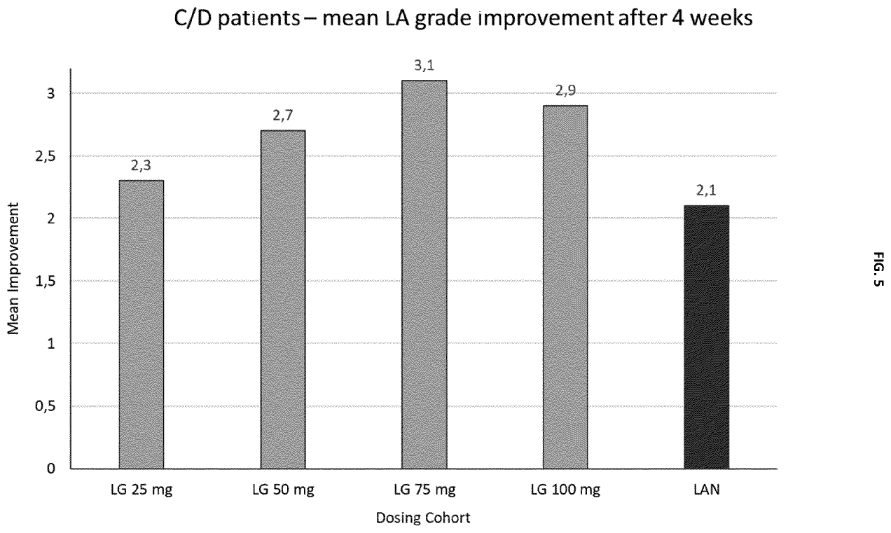

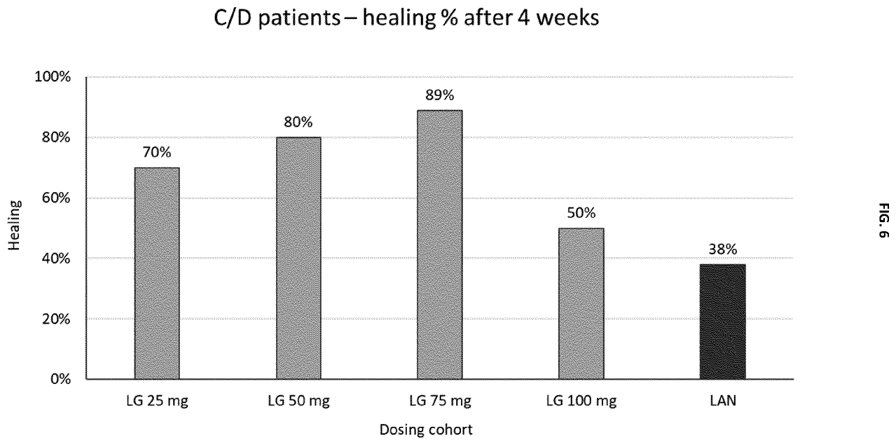

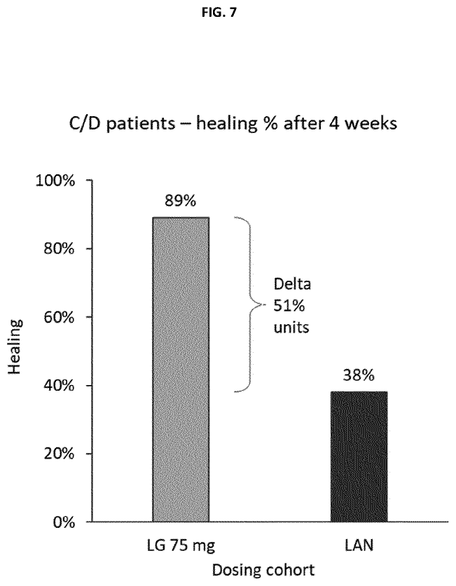

The results show a clear dose response in the healing rates. For patients with moderate to severe eGERD (LA grade C/D), the highest mean LA grade improvement after 4 weeks in a linaprazan glurate dosing group was 3.1, compared to 2.1 in the lansoprazole group (see FIG. 5). The LA grade improvement of all LA grade C/D patients treated with linaprazan glurate was higher than lansoprazole. The highest four-week healing rate in a linaprazan glurate dosing group was 89%, compared to 38% in the lansoprazole group; see FIG. 6 (all LG dose group) and FIG. 7 (LG 75 mg dose group). The response in the linaprazan glurate 100 mg dose group was lower than expected, but this group comprised a higher proportion of the most severe (“hard to treat”) LA grade D patients than the other dose groups. In addition, for 6 out of 7 patients in the LG 100 mg dose group, the 4-week LA grade was A, i.e., these patients failed on the finish line.

While the study was not powered to demonstrate significance towards the comparator lansoprazole, the healing rate of all LA grade C/D patients treated with linaprazan glurate was significantly higher than lansoprazole in a post-hoc analysis (Fisher's exact test, mean harmonic p-value=0.0404).

For patients with milder eGERD (LA grade A/B), the highest four-week healing rate in a linaprazan glurate dosing group was 91%, compared to 81% in the lansoprazole group.

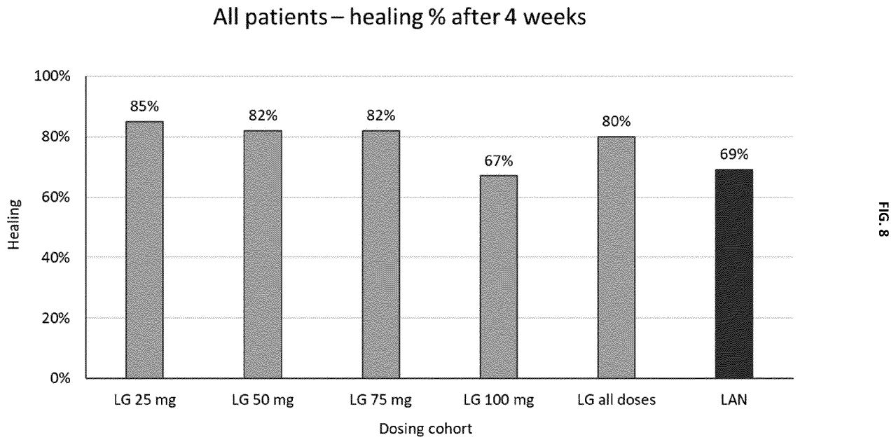

For all patients treated with linaprazan glurate (LA grade A/B/C/D), the mean healing rate was 80% compared to 69% in the lansoprazole treated group; see FIG. 8.

### Heartburn

Reflux related symptoms were assessed based on a modified RESQ-eDiary (mRESQ-eD). Patients were asked to report every 12-hours, once before the morning dose and once before the evening dose of the Investigational Medicinal Product (IMP). Results for erosive patients of all LA grades are shown in Table 4.

TABLE 4
Heartburn during week 4 (erosive patients, all LA grades)

| | LG 25 mg | LG 50 mg | LG 75 mg | LG 100 mg | LAN 30 mg |
|---|---|---|---|---|---|
| | (N = 21) | (N = 27) | (N = 20) | (N = 19) | (N = 20) |
| % heartburn-free 24-hour days | 52 | 56 | 56 | 76 | 52 |
| (mean) | | | | | |
| % at most mild symptoms 24-hour | 96 | 96 | 97 | 100 | 88 |
| days (mean) | | | | | |
| Patients (%) totally free from | 45 | 44 | 50 | 63 | 32 |
| heartburn | | | | | |
| Patients (%) with at most mild | 97 | 97 | 87 | 92 | 79 |
| heartburn | | | | | |
| Patients (%) with non-mild | 6 | 6 | 13 | 8 | 21 |
| heartburn | | | | | |

### Full Analysis Set (FAS)

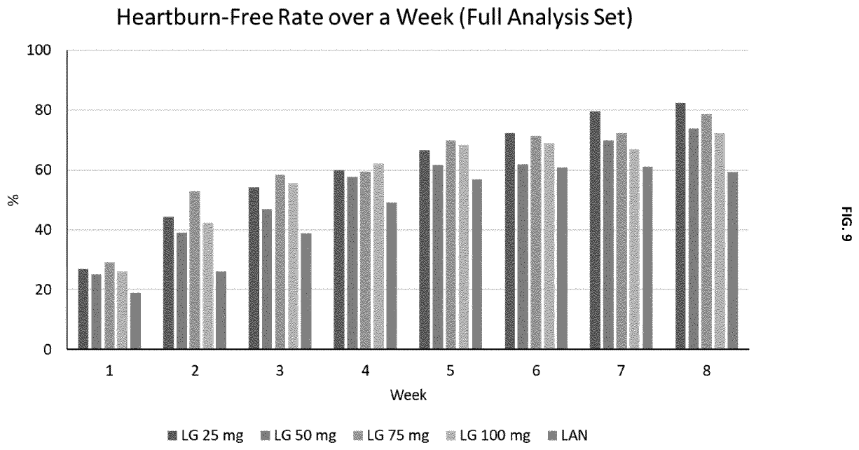

A chart of the mRESQ-eD assessed heartburn-free 24-hour days over a week for all patients is shown in FIG. 9.

### Double-Blind Period (Week 1 to 4)

Overall, the severity of heartburn for FAS over the first week of treatment was mild, at most 1.91 (Min 0=Did not have; Max 5=Severe). Patients receiving lansoprazole reported the highest severity grade of heartburn both morning and evening, and patients receiving LG 75 mg reported the lowest severity grade both morning and evening.

All linaprazan glurate treatment groups reported more heartburn-free days week 1 to week 4 compared to the lansoprazole treatment. The LG 75 mg treatment group reported the highest percentage of symptom free days (29.2% at week 1, 52.9% at week 2, and 58.46.56% at week 3), while at week 4, the LG 100 mg treatment group reported the highest percentage (62.2%).

Open-label period (week 5 to 8; all patients treated with lansoprazole 30 mg QD) All linaprazan glurate treatment groups reported more heartburn-free days week 5 to week 8 compared to lansoprazole. At week 5, the LG 75 mg treatment group reported the highest percentage of heartburn-free days (69.6%). From week 6 through week 8, the highest percentage of heartburn-free days was reported for the LG 25 mg treatment group (72.31.66% at week 6, 79.4% at week 7, and 82.4% at week 8).

### FAS Erosive

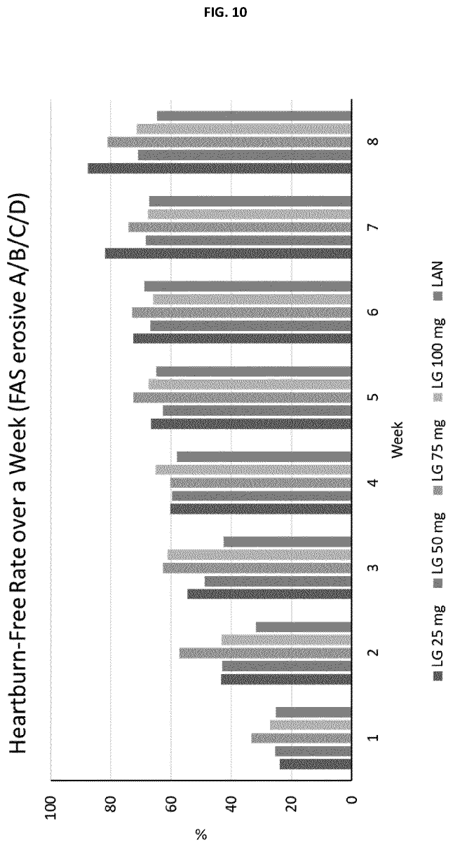

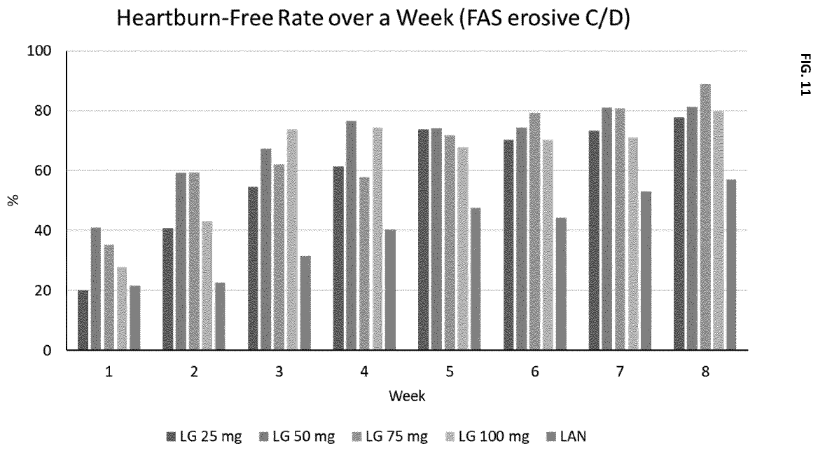

A chart of the mRESQ-eD assessed heartburn-free 24-hour days over a week for all LA grade A/B/C/D patients is shown in FIG. 10, and the corresponding chart for LA grade C/D patients is shown in FIG. 11.

### Double-Blind Period (Week 1 to 4)

Overall, the severity of heartburn for FAS over the first week of treatment was mild, at most 1.75 (Min 0=Did not have; Max 5=Severe). Patients receiving lansoprazole reported the highest heartburn severity both morning and evening, and patients receiving LG 75 mg reported the lowest heartburn severity morning and evening.

All linaprazan glurate treatment groups reported more heartburn-free days week 1 to week 4 compared to the lansoprazole treatment. The LG 75 mg treatment group reported the highest percentage of symptom free days week 1-3 (33.3% at week 1, 57.2% at week 2, and 62.6% at week 3), while at week 4, the LG 100 mg treatment group reported the highest percentage (65.2%).

For LA Grade A/B the highest percentage of heartburn-free days was reported by treatment group LG 75 mg at week 1 to 3, while at week 4 lansoprazole reported the most heartburn-free days.

For LA Grade C/D, the highest percentage of heartburn-free days were by treatment group LG 50 mg at week 1 and 4, by LG 75 mg at week 2 and by LG 100 mg at week 3. The lansoprazole group reported the lowest percentage of heartburn-free days week 2-4.

Open-Label Period (Week 5 to 8; all Patients Treated with Lansoprazole 30 mg QD)

All linaprazan glurate treatment groups reported more heartburn-free days week 5 to week 8 compared to the lansoprazole treatment group. At week 5 and week 6, the LG 75 mg treatment group reported the highest percentage of heartburn-free days (72.6% and 73.0%, respectively), while at week 7 and week 8 the highest percentage of heartburn-free days was reported for the LG 25 mg treatment group (81.9% and 87.6%, respectively).

For LA Grade A/B, the highest percentage of heartburn-free days was reported by treatment group LG 75 mg at week 5, lansoprazole reported the highest at week 6 and treatment group LG 25 mg reported the highest percentage of heartburn-free days at weeks 7 and 8.

For LA Grade C/D, the highest percentage of heartburn-free days were reported by LG 50 mg at week 5 and by LG 75 mg week 6 to 8. The lansoprazole group reported the lowest percentage of heartburn-free days week 5-8.

### Safety

Linaprazan glurate was generally well tolerated and safety data was comparable to that of lansoprazole. The numbers of adverse events were similar between study groups and there were no unexpected findings. Throughout the study 103 adverse events (AEs) were reported in 58 patients, with a similar distribution between the different dose levels. No treatment-related serious AEs were reported. The most common treatment emergent adverse event (TEAE) reported was COVID-19 infection, which occurred in 4% of the total study population. See Table 5.

TABLE 5
TEAE occurring in ≥2% of study population in total or >2 patients in any dose level

| | LG 25 mg | LG 50 mg | LG 75 mg | LG 100 mg | LAN 30 mg | Total |
|---|---|---|---|---|---|---|
| COVID-19 | 1 (2.0) | 2 (4.2) | 1 (1.9) | 4 (8.5) | 2 (4.0) | 10 (4.0) |
| events (%) | | | | | | |
| Headache | 1 (2.0) | 3 (6.3) | 1 (1.9) | 1 (2.1) | 1 (2.0) | 7 (2.8) |
| events (%) | | | | | | |
| Nausea | 1 (2.0) | 0 (0.0) | 0 (0.0) | 0 (0.0) | 4 (8.0) | 5 (2.0) |
| events (%) | | | | | | |
| Constipation | 2 (3.9) | 1 (2.1) | 2 (3.8) | 0 (0.0) | 0 (0.0) | 5 (2.0) |
| events (%) | | | | | | |

1. A method of treating or preventing a gastrointestinal inflammatory or a gastric acid related disease, comprising administering linaprazan glurate, or a pharmaceutically acceptable salt thereof, orally to a subject in an amount of from about 25 to about 100 mg twice daily.

2. The method of claim 1, wherein linaprazan glurate, or a pharmaceutically acceptable salt thereof, is administered in an amount of about 25 mg.

3. The method of claim 1, wherein linaprazan glurate, or a pharmaceutically acceptable salt thereof, is administered in an amount of about 50 mg.

4. The method of claim 1, wherein linaprazan glurate, or a pharmaceutically acceptable salt thereof, is administered in an amount of about 75 mg.

5. The method of claim 1, wherein linaprazan glurate, or a pharmaceutically acceptable salt thereof, is administered in an amount of about 100 mg.

6-7. (canceled)

8. The method of claim 1, wherein linaprazan glurate, or a pharmaceutically acceptable salt thereof, is administered for at least 1 week.

9. The method of claim 1, wherein linaprazan glurate, or a pharmaceutically acceptable salt thereof, is administered for at least 4 weeks.

10. The method of claim 1, wherein the gastrointestinal inflammatory or gastric acid related disease is gastroesophageal reflux disease (GERD) or erosive gastroesophageal reflux disease (eGERD).

11. The method of claim 1, wherein the GERD or eGERD is LA grade A.

12. The method of claim 1, wherein the GERD or eGERD is LA grade B.

13. The method of claim 1, wherein the GERD or eGERD is LA grade C.

14. The method of claim 1, wherein the GERD or eGERD is LA grade D.

15. The method of claim 1, wherein the subject, following oral administration of linaprazan glurate, or a pharmaceutically acceptable salt thereof, exhibits an improvement of the LA grade of reflux esophagitis.

16. The method of claim 1, wherein the eGERD is LA grade A and wherein the improvement of the LA grade is 1 step.

17. The method of claim 15, wherein the eGERD is LA grade B and wherein the improvement of the LA grade is 1 or 2 steps.

18. The method of claim 15, wherein the eGERD is LA grade C and wherein the improvement of the LA grade is 2 or 3 steps.

19. The method of claim 15, wherein the eGERD is LA grade D and wherein the improvement of the LA grade is 3 or 4 steps.

20. The method of claim 1, wherein the subject, following oral administration of linaprazan glurate, or a pharmaceutically acceptable salt thereof, exhibits an increase in heartburn-free 24-hour days.

21. The method of claim 1, wherein the subject, following oral administration of linaprazan glurate, or a pharmaceutically acceptable salt thereof, has at least about 55%, at least about 60%, at least about 65%, at least about 70%, or at least about 75% heartburn-free 24-hour days over a week.

22. A method of treating or preventing a gastrointestinal inflammatory or a gastric acid related disease, comprising administering linaprazan glurate, or a pharmaceutically acceptable salt thereof, orally, to a subject in an amount of from about 25 to about 100 mg twice daily, wherein following such oral administration the subject exhibits a minimum plasma concentration (Cmin) of linaprazan of at least 240 nmol/L.

23. The method of claim 1, wherein linaprazan glurate is present as a crystalline salt of linaprazan glurate.

24. The method of claim 23, wherein linaprazan glurate is present as a crystalline hydrochloride salt of linaprazan glurate.

25. The method of claim 24, wherein the crystalline hydrochloride salt of linaprazan glurate is an anhydrate.

26. The method of claim 25, wherein the anhydrate of the hydrochloride salt of linaprazan glurate is Form 1.

27. The method of claim 26, wherein Form 1 has an XRPD pattern, obtained with CuKα1-radiation, with at least two peaks at ° 2θ values selected from the list consisting of 9.1±0.2, 13.8±0.2, 14.0±0.2, 20.0±0.2, 22.9±0.2, 23.4±0.2, 24.4±0.2, 24.6±0.2 and 26.7±0.2.
